(19) United States
(12) Patent Application Publication
(10) Pub. No.: US 2004/0019096 A1
(43) Pub. Date: Jan. 29, 2004

(54) Novel formulations of carvedilol

(76) Inventors: Vlassios Andronis, King of Prussia, PA (US); Kimberly A Lamey, King of Prussia, PA (US); Choon K Oh, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,671

(22) PCT Filed: Oct. 23, 2001
(86) PCT No.: PCT/US01/50872

(51) Int. Cl. A61K 31/403; A61K 31/19

(52) U.S. Cl. 514/411; 514/566; 514/574

(57) ABSTRACT

This invention relates to a novel formulations comprising carvedilol and methods of using these formulations to treat hypertension, congestive heart failure and angina.

### FIELD OF THE INVENTION

This invention relates to novel formulations of carvedilol and to the use of such formulations in the treatment of hypertension, congestive heart failure and angina.

### BACKGROUND OF THE INVENTION

The compound, 1-(carbazol4-yloxy-3-[[2-(o-methoxyphenoxy)ethyl]amino]-2-propanol, is known by the name “carvedilol” and is the subject of U.S. Pat. No. 4,503,067 (the '067 patent), issued Mar. 5, 1985. This compound has the following structure:

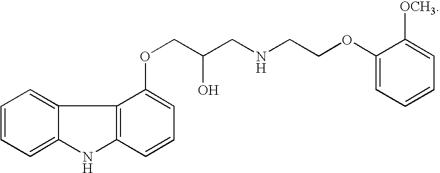

Carvedilol is useful in the treatment of hypertension, congestive heart failure and angina

The current commercial formulation for carvedilol is immediate release, and it is administered twice daily. The immediate release formulation of carvedilol is rapidly and extensively absorbed following oral administration, with the terminal elimination half-life ranging between 7-10 hours. A once-daily dosing formulation for carvedilol is commercially desirable, would simplify a patient's dosing regimen and may improve compliance rates. Thus, it is an object of the instant invention to develop a once-daily dosing formulation for carvedilol.

According to the instant invention, it has been found that carvedilol can be formulated in novel formulations for once-daily dosing.

### SUMMARY OF THE INVENTION

The present invention provides for the use of a pharmaceutically acceptable organic acid in formulations comprising carvedilol.

This invention also provides for the use of such formulations for the treatment of hypertension, congestive heart failure and angina.

### DESCRIPTION OF THE INVENTION

According to the present invention, compositions of carvedilol are provided in spray-dried powder form or standard drug substance form. The spray-dried powder compositions are prepared using a process that involves wet milling. The suspension, thus produced, is spray dried using a spray dryer or granulated using a fluid bed granulator. The composition may then be formulated, for example, in the form of tablets or capsules. Orally administrated formulations are preferred.

Importantly, the present invention provides for a formulation comprising carvedilol, which further comprises of a pharmaceutically acceptable organic acid.

As used herein, the term “pharmaceutically acceptable organic acid” refers to organic acids which are without pharmacological effect per se, have acceptable organoleptic properties, have acceptable density, do not have an extreme pH and are preferably solid. Examples include mono-carboxylic acids and poly-carboxylic acids having from 2 to 25, preferably from 2 to 10, carbon atoms; monocyclic and polycyclic aryl acids, such as benzoic acid; as well as monohydrogen, dihydrogen and metal salts of multi-valent acids. A single pharmaceutically acceptable organic acid may be used, or two or more of such may be used in combination. Preferably, the organic acid is a C₍₂₋₁₀₎alkyl- or alkenyl-carboxylic acid having from one, two or three carboxylic acid groups, and optionally with one or more hydroxy substituents or an additional CO group in the carbon chain, for instance malonic acid, succinic acid, fumaric acid, maleic acid, adipic acid, lactic acid, levulinic acid, sorbic acid, glutamic acid, aspartic acid, oleic acid, glutaric acid, glycine, arginine or a fruit acid, such as tartaric acid, malic acid, ascorbic acid or citric acid, most preferably citric acid.

The wet milling process of the present invention is well known to those skilled in the art and is described in:

J. A. Herbst and J. L. Sepulveda “Fundamentals of Fine and Ultrafine Grinding in a Stirred Ball Mill” International Powder and Bulk Solids Handling and Processing, 452, May 1978 and

L. Y. Sadler III, D. A. Stanley, and D. R. Brooks “Attrition Mill Operational Characteristics” Powder Technology 12 (1975) 19-28. Spray drying of milled compositions is carried out most suitably using a spray dryer, such as Yamato GA-32 Spray Dryer [Yamato Scientific America Inc., Orangeburg, N.Y.]. Alternately, granulation of milled compositions is carried out most suitably using a fluid bed granulator, such as Glatt fluid bed granulator, or a high shear granulator.

The spray-dried powder, thus produced, is then used in tablets for oral administration in a unit dose. These oral tablets comprise conventional controlled release formulations, such as tablets, having a sustained release or an enteric coating, or otherwise modified to control the release of the active compound, for example by the inclusion of gel forming polymers or matrix forming waxes.

Additionally, carvedilol drug substance is mixed with an organic acid in either solution or suspension to form a drug medium to subsequently layer onto pellets or granules. The drug layered pellets or granules are then coated to consist of a standard seal coat, enteric coat or a sustained release coat permeable to gastrointestinal juices. The controlled release formulations are prepared, for example, as described in U.S. Pat. No. 4,524,060, issued Jun. 18, 1985, and U.S. Pat. No. 4,983,401, issued Jan. 8, 1991. Other controlled release formulations are described in U.S. Pat. No. 4,880,830, issued Nov. 14, 1989, and U.S. Pat. No. 5,068,112, issued Nov. 26, 1991.

The controlled release formulations containing carvedilol and organic acid may also be in the form of a non-compressed drug layered pellet loaded into a standard capsule. This capsule is then enteric coated for delayed release and then subsequently coated with an immediate release portion of Carvedilol for a two burst system.

Tablets or capsules for oral administration are usually presented in a unit dose, and contain conventional excipients such as binding agents, fillers and diluents (tableting or compression aids), lubricants, disintegrants, colorants, flavourings, and wetting agents. The tablets may be coated according to techniques well known in the art.

These oral formulations may be prepared by conventional methods of blending, filling, tableting, or the like. Repeated blending operations may be used to distribute the active agent throughout those compositions employing large quantities of fillers. Such operations are, of course, well known in the art.

Thus, the present invention provides for the use of a pharmaceutically acceptable organic acid in the formulations comprising carvedilol. The formulation is adapted for oral administration. The formulation is presented as a unit dose. Such a formulation is taken once daily. The preferred unit dosage forms include tablets or capsules comprising 25 mg or 50 mg of carvedilol; however, the present invention also includes doses from 6.25 mg to 100 mg.

No unacceptable toxicological effects are expected when carvedilol is administered in accordance with the present invention. The following examples are illustrative of the instant invention. These examples are not intended to limit the scope of this invention as defined herein above and as claimed herein below.

### EXAMPLES 1 & 2

### Carvedilol 50 mg Controlled Release Aqueous Film Coated Tablets

The carvedilol 50 mg controlled release (CR) aqueous film coated (AFC) tablets were prepared from a carvedilol spray-dried powder which was blended with external excipients and a lubricant, compressed, and finally coated with a clear aqueous film coat followed by a Eudragit®-based coat. Product CE tablets were made with fumaric acid, whereas Product CF tablets are made with citric acid.

TABLE 1
Unit Formulas for Carvedilol 50 mg CR AFC Tablet, Products CE and CF

| | Quantity |
|---|---|
| Ingredients | (mg/Tablet) |
| “Carvedilol Spray-Dried Powder” consisting of: | (78.06¹) |
| Carvedilol | 52.07¹ |
| Poloxamer 407 | 10.38¹ |
| Povidone | 5.23¹ |
| Hydroxyethyl Cellulose | 10.38¹ |
| Purified Water | q.s.² |
| Fumaric Acid for Product CE | 120.00 |
| or Citric Acid (monohydrate) for Product CF | |
| Dibasic Calcium Phosphate Dihydrate | 155.94¹ |
| Microcrystalline Cellulose | 174.00 |
| Crospovidone | 60.00 |
| Silicon Dioxide Colloidal | 6.00 |
| Magnesium Stearate | 6.00 |
| Total Weight Tablet Core | 600.00 |
| Clear Opadry YS-1-19025-A | 12.00 |
| Aqueous Dispersion of Methacrylic Acid | 45.58 |
| Copolymer, Type C (Eudragit ® L30 D-55) | |
| Triethyl Citrate | 5.83 |
| Sodium Lauryl Sulfate | 1.06 |
| Glyceryl Stearate (Imwitor 900 Powder) | 0.53 |
| Purified Water | q.s.³ |
| Total Weight Coated Tablet | 665.00 |

¹Based on 96% label claim for the “Carvedilol Spray-Dried Powder.” These levels will change based upon the % label claim achieved during the spray drying process and are adjusted during the blending step.
²Removed during spray drying process.
³Removed during drying.

### EXAMPLES 3 & 4

### Carvedilol 50 mg Controlled Release Matrix Tablets

The carvedilol controlled release (CR) matrix tablets (Products CG and CH) are prepared from a carvedilol granulation containing either a carvedilol spray-dried powder or standard carvedilol drug substance, respectively. A citric acid granulation is prepared separately. The desired carvedilol granulation and the citric acid granulation are blended together along with external excipients and finally a lubricant to produce the mix from which tablets are then compressed.

TABLE 2
Unit Formulas for Carvedilol 50 mg CR Matrix Tablets,
Products CG and CH

| | Quantity (mg/Tablet) | |
|---|---|---|
| Ingredients | CG | CH |
| Carvedilol Granulation | | |
| “Carvedilol Spray-Dried Powder” | (78.21⁴) | |
| consisting of: | | |
| Carvedilol | 52.17⁴ | — |
| Poloxamer 407 | 10.40⁴ | — |
| Povidone | 5.24⁴ | — |
| Hydroxyethyl Cellulose | 10.40⁴ | — |
| Purified Water | q.s.⁵ | — |
| Carvedilol | — | 50.00 |
| Hydroxypropyl Methylcellulose | 77.01 | 95.96 |
| Carboxymethylcellulose Sodium | 38.30 | 47.57 |
| Povidone | 8.07 | 8.06 |
| Purified Water | q.s.⁵ | q.s.⁵ |
| Citric Acid Granulation | | |
| Citric Acid | 104.98 | 104.98 |
| Hydroxypropyl Methylcellulose | 54.43 | 54.43 |
| Carboxymethylcellulose Sodium | 27.22 | 27.22 |
| Povidone | 7.78 | 7.78 |
| Purified Water | q.s.⁵ | q.s.⁵ |
| Final Blend | | |
| Microcrystalline Cellulose | 192.00⁴ | 192.00⁴ |
| Silicon Dioxide Colloidal | 6.00 | 6.00 |
| Magnesium Stearate | 6.00 | 6.00 |
| Total Tablet Weight | 600.00 | 600.00 |

⁴Based on 96% label claim for the “Carvedilol Spray-Dried Powder.” These levels will change based upon the % label claim achieved during, the spray drying process and are adjusted during the blending step.
⁵Removed during processing.

### EXAMPLE 5

### Carvedilol 25 mg Controlled Release Capsule

The carvedilol controlled release (CR) capsule is prepared from carvedilol drug layered pellets containing standard carvedilol drug substance, aspartic acid and Opadry Clear. The drug layered pellets are then coated with a sustained release coat, Aquacoat ECD-30, and filled into standard capsule shells for administration process.

| Ingredients | (mg/Capsule) |
|---|---|
| “Carvedilol Drug Layered Pellets” consisting of: | (480.7) |
| Carvedilol | 25.0 |
| L-Aspartic Acid | 31.2 |
| Opadry Clear | 25.0 |
| Purified Water | q.s.¹ |
| Microcrystalline Cellulose Spheres | 399.5 |
| Ethylcellulose Aqueous Dispersion (Aquacoat ECD-30) | 113.9 |
| Dibutyl Sebacate | 36.1 |
| Purified Water | q.s.¹ |
| Opadry Clear | 1.9 |
| Purified Water | q.s.¹ |
| Size 00 Capsule Shell | 118.0 |
| Total Weight Capsule | 750.6 |

¹Removed during drug layering or coating process.

### EXAMPLE 6

### Carvedilol 25 mg Controlled Release Capsule

The carvedilol controlled release (CR) capsule is prepared from carvedilol drug layered pellets containing standard carvedilol drug substance, aspartic acid and Opadry Clear. 22.5 mg strength drug layered pellets are filled into standard capsule shells. The capsules are enteric coated for the delayed release portion and a 2.5 mg strength immediate release top-coat is then applied for the initial burst effect.

| Ingredients | (mg/Capsule) |
|---|---|
| “Carvedilol Drug Layered Pellets” consisting of: | (432.7) |
| Carvedilol | 22.5 |
| L-Aspartic Acid | 28.1 |
| Opadry Clear | 22.5 |
| Purified Water | q.s¹ |
| Microcrystalline Cellulose Spheres | 359.6 |
| Aqueous Dispersion of Methacrylic Acid Copolymer, | 49.7 |
| Type C (Eudragit ® L30 D-55) | |
| Triethyl Citrate | 5.8 |
| Silicon Dioxide Colloidal | 4.2 |
| Sodium Lauryl Sulfate | 0.4 |
| Purified Water | q.s.¹ |
| Carvedilol | 2.5 |
| L-Aspartic Acid | 0.2 |
| Opadry Clear | 2.5 |
| Purified Water | q.s.¹ |
| Size 1 Capsule Shell | 76.0 |
| Total Weight Capsule | 574.0 |

¹Removed during drug layering or coating process.

in vivo Pharmacokinetic Evaluation of Formulations

The bioavailability of the formulations according to the present invention are evaluated in healthy human volunteers. The study is an open-label, single dose, randomized, four-period, incomplete block, crossover study. Each subject receives a single dose of the immediate release formulation in addition to single oral doses of 3 of the 4 controlled-release formulations according to a randomization schedule. The regimens for the study are tabulated below:

| Product | Description |
|---|---|
| CE | 50 mg carvedilol in a controlled-release, Enteric I tablet |
| | formulation |
| CF | 50 mg carvedilol in a controlled-release, Enteric II tablet |
| | formulation |
| CG | 50 mg carvedilol in a controlled-release, Matrix I tablet |
| | formulation |
| CH | 50 mg carvedilol in a controlled-release, Matrix II tablet |
| | formulation |
| CI | 2 × 25 mg carvedilol immediate release tablets, commercial |
| | formulation |

Pharmacokinetic sampling for measurement of plasma carvedilol concentrations is conducted over a 48-hour period following administration of study medication in each study session. There is a washout period of at least 7 days between dosing in sessions. Female subjects return 7-10 days following dosing in the last study session for a follow-up pregnancy test. The total duration (from screening to end of the study) of each subject's participation will be five to eight weeks.

The primary endpoint is the AUC of carvedilol. Secondary endpoints include Cmax, Tmax, and T½, as data permit.

It is to be understood that the invention is not limited to the embodiments illustrated hereinabove and the right is reserved to the illustrated embodiments and all modifications coming within the scope of the following claims. The various references to journals, patents and other publications which are cited herein comprise the state of the art and are incorporated herein by reference as though fully set forth.

What is claimed is:

1. A matrix formulation comprising carvedilol and a pharmaceutically acceptable organic acid.

2. The formulation according to claim 1 wherein the pharmaceutically acceptable organic acid is citric acid.

3. An enteric coated formulation comprising carvedilol and a pharmaceutically acceptable organic acid.

4. The formulation according to claim 3 wherein the pharmaceutically acceptable organic acid is citric acid.

5. A drug layered formulation comprising carvedilol and a pharmaceutically acceptable organic acid.

6. The formulation according to claim 5 wherein the pharmaceutically acceptable organic acid is aspartic acid.

7. A method of treating hypertension, congestive heart failure or angina which comprises administering to a subject in need thereof an effective amount of the formulation according to any one of claims 1-6.
